(19) United States
(12) Patent Application Publication
(10) Pub. No.: US 2025/0268815 A1
(43) Pub. Date: Aug. 28, 2025

(54) TRANSDERMAL DELIVERY SYSTEMS FOR ADMINISTRATION OF HYDROPHILIC MOLECULES AND METHODS OF MAKING AND USING SAME

(71) Applicant: STATE OF ISRAEL, MINISTRY OF AGRICULTURE & RURAL DEVELOPMENT ARO (VOLCANI INSTITUTE), Rishon Lezion (IL)

(72) Inventors: Elena POVERENOV, Nes Ziona (IL); Guy COHEN, Dead Sea (IL)

(21) Appl. No.: 18/687,955

(22) PCT Filed: Aug. 29, 2022
(86) PCT No.: PCT/IL2022/050938

§ 371 Date: Feb. 29, 2024

Related U.S. Application Data
(60) Provisional application No. 63/238,796, filed on Aug. 31, 2021.

(51) Int. Cl. A61K 9/00; A61K 38/28; A61K 47/38; A61K 47/44

(52) U.S. Cl. CPC A61K 9/0014; A61K 9/0002; A61K 38/28; A61K 47/38; A61K 47/44

(57) ABSTRACT

A transdermal delivery system including: a) one or more N-alkylamidated CMCs (carboxymethylcellulose); b) a hydrophilic active agent; and c) a lipophilic carrier. Related methods of manufacture and use are also disclosed.

### RELATED APPLICATIONS

This PCT application claims the benefit according to 35 U.S.C. § 119(e) of U.S. provisional patent application 63/238,796 filed on Aug. 31, 2021 entitled “Biocompatible systems for effective transdermal delivery of insulin based on flexible self-adjusting N-alkylamidated carboxymethyl cellulose derivatives” and having the same inventors as the present application; which is fully incorporated herein by reference.

### FIELD OF THE INVENTION

The invention is in the field of transdermal drug delivery.

### BACKGROUND OF THE INVENTION

Insulin is a peptide hormone produced in the pancreas. Insulin is the main anabolic hormone and regulates the metabolism of carbohydrate, fat and proteins by promoting the absorption of glucose from the blood into the liver. The absorbed glucose is converted into glycogen and/or fats. High concentrations of insulin in the blood prevent release of glucose by the liver. Low concentrations of insulin in the blood promote widespread catabolism, especially of reserve body fat. Insulin is a hydrophilic macromolecule with a size of 5.5-6.0 kDa.

Insulin deficiency causes diabetes which is most commonly treated by insulin injection.

Carboxymethyl cellulose (CMC) or cellulose gum is a cellulose derivative with carboxymethyl groups (—CH₂—COOH) bound to hydroxyl groups of the monomers of the cellulose backbone. CMC is commonly used in the food industry as a thickening agent and in the pharmaceutical industry as a disintegrant.

### SUMMARY OF THE INVENTION

One aspect of some embodiments of the invention relates to modification of carboxymethylcellulose (CMC) by N-alkylamidation using an appropriate amine. In some embodiments hexyl amine and/or dodecyl amine are used for N-alkylamidation. Throughout this specification and the accompanying claims CMC-6 refers to CMC N-alkylamidated with hexyl amine and CMC-12 refers to CMC N-alkylamidated with dodecyl amine. In other exemplary embodiments of the invention, other amines are employed for N-alkylamidation. According to various exemplary embodiments of the invention N-alkylamidation contributes to an ability of the modified CMC molecule to solubilize hydrophilic compounds in a lipid environment. One possible application of this property is transdermal delivery of insulin (which is hydrophilic).

For purposes of this specification and the accompanying claims, the term “hydrophilic” indicates solubility in water of at least 50 g/liter.

According to another aspect of some embodiments of the invention, a transdermal delivery system includes an N-alkylamidated CMC, a lipophilic carrier and a hydrophilic active agent to be delivered. According to various exemplary embodiments of the invention the delivery system is provided as a cream, lotion, ointment or patch. In some exemplary embodiments of the invention, the system includes several different N-alkylamidated CMCs, each with a different absorption profile relative to skin. In some embodiments use of several different N-alkylamidated CMCs, each with a different absorption profile relative to skin contributes to an ability of the system to achieve sustained release of the hydrophilic active agent over a period of time. In some embodiments the hydrophilic active agent is insulin.

According to another aspect of some embodiments of the invention, a treatment method includes application of a hydrophilic active agent to a skin surface in a carrier including one or more N-alkylamidated CMCs and a lipophilic diluent. In some embodiments the active agent is insulin. Alternatively or additionally, in some embodiments the N-alkylamidated CMCs include CMC-12.

It will be appreciated that the various aspects described above relate to solution of technical problems associated with transfer of hydrophilic molecules across the skin.

Alternatively or additionally, it will be appreciated that the various aspects described above relate to solution of technical problems related to administration of insulin without a needle.

In some exemplary embodiments of the invention there is provided a transdermal delivery system including: a) one or more N-alkylamidated CMCs (carboxymethylcellulose); b) a hydrophilic active agent; and c) a lipophilic carrier. In some embodiments the hydrophilic active agent includes insulin. Alternatively or additionally, in some embodiments the transdermal delivery system is provided in a form selected from the group consisting of a cream, a lotion, an ointment and a patch. Alternatively or additionally, in some embodiments the transdermal delivery system is provided in a sustained release configuration. Alternatively or additionally, in some embodiments the transdermal delivery system is provided in an immediate release configuration. Alternatively or additionally, in some embodiments the one or more N-alkylamidated CMCs includes two or more N-alkylamidated CMCs. Alternatively or additionally, in some embodiments the one or more N-alkylamidated CMCs includes CMC-12.

In some exemplary embodiments of the invention there is provided a method of treatment including applying a hydrophilic active agent to a skin surface in a carrier including one or more N-alkylamidated CMCs and a lipophilic diluent. In some embodiments the hydrophilic active agent includes insulin. Alternatively or additionally, in some embodiments the applying includes contacting an item selected from the group consisting of a cream, a lotion, an ointment and a patch to the skin. Alternatively or additionally, in some embodiments the one or more N-alkylamidated CMCs includes two or more N-alkylamidated CMCs. Alternatively or additionally, in some embodiments the one or more N-alkylamidated CMCs includes CMC-12.

In some exemplary embodiments of the invention there is provided a composition includes insulin in a carrier including one or more N-alkylamidated CMCs and a lipophilic diluent for use in treatment of diabetes. In some embodiments the composition is formulated as an item selected from the group consisting of a cream, a lotion, an ointment and a patch. Alternatively or additionally, in some embodiments the one or more N-alkylamidated CMCs includes two or more N-alkylamidated CMCs. Alternatively or additionally, in some embodiments the one or more N-alkylamidated CMCs includes CMC-12.

In some exemplary embodiments of the invention there is provided a method of manufacture includes: (a) dissolving carboxymethylcellulose (CMC) in water to form a CMC solution; (b) dissolving 1-(3-Dimethylaminopropyl)-3-ethylcarbodiimide hydrochloride (EDC) and N-hydroxysuccinimide (NHS) in the CMC solution; and (c) adding an amine to initiate N-alkylamidation of the CMC and produce a modified CMC solution. In some embodiments the amine includes hexyl amine and the N-alkylamidation produces CMC-6. Alternatively or additionally, in some embodiments the amine includes dodecyl amine and the N-alkylamidation produces CMC-12. Alternatively or additionally, in some embodiments

The EDC and the NHS are provided in equimolar amounts. Alternatively or additionally, in some embodiments the EDC and the NHS and the amine are provided in equimolar amounts. Alternatively or additionally, in some embodiments the method includes dissolving a hydrophilic active agent in the modified CMC solution. Alternatively or additionally, in some embodiments the hydrophilic active agent includes insulin. Alternatively or additionally, in some embodiments the method includes mixing a lipophilic diluent with the modified CMC solution.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, integers, actions or components without precluding the addition of one or more additional features, integers, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The phrase “adapted to” as used in this specification and the accompanying claims imposes additional structural limitations on a previously recited component.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

Percentages (%) are W/W (weight per weight) unless otherwise indicated.

For purposes of this specification and the accompanying claims, the terms “lipophilic carrier” and “lipophilic diluent” include, but are not limited to oils (e.g. sunflower oil; SO).

In experimental examples presented hereinbelow, insulin is used as an exemplary hydrophilic active agent. Other exemplary hydrophilic active agents include, but are not limited to, other peptide hormones such as adrenocorticotropic hormone (ACTH), adropin, amylin, angiotensin, atrial natriuretic peptide (ANP), calcitonin, cholecystokinin (CCK), gastrin, ghrelin, glucagon, growth hormone, follicle-stimulating hormone (FSH), leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, also called arginine vasopressin (AVP) or anti-diuretic hormone (ADH) and vasoactive intestinal peptide (VIP).

### BRIEF DESCRIPTION OF THE DRAWINGS

In order to understand the invention and to see how it may be carried out in practice, embodiments will now be described, by way of non-limiting example only, with reference to the accompanying figures. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1a is a diagrammatic representation of the Synthesis of N-alkylamidated carboxymethyl cellulose derivatives, referred to as CMC-6 and CMC-12;

FIG. 1b is histogram of 1H-NMR 400 MHZ showing arbitrary units as a function of f1(ppm) for CMC; CMC-6 and CMC-12 as indicated;

FIG. 1c is histogram of ATR-FTIR showing % Transmittance as a function of Wavenumber [cm⁻¹] for CMC; CMC-6 and CMC-12 as indicated;

FIG. 2a is histogram of Thermal gravimetric analysis (TGA) in weight loss [w/w %] as a function of temperature in ° C. for CMC; CMC-6 and CMC-12 as indicated;

FIG. 2b is histogram of differential scanning calorimetry (DSC) showing unsubtracted heat flow [mW] as a function of temperature in ° C. for CMC; CMC-6 and CMC-12 as indicated;

FIG. 3a is a Transmission Electron Microscopy (TEM) image of CMC-6 with Scale bar set to 200 nm;

FIG. 3b is a Transmission Electron Microscopy (TEM) image of CMC-12 with Scale bar set to 200 nm;

FIG. 4a is a histogram of Florescence emission spectra showing intensity [a.u.] as a function of wavelength [nm] for sunflower oil (SO), Insulin, Insulin+CMC, Insulin+CMC-6 and Insulin+CMC-12;

FIG. 4b is a confocal-laser-scanning-microscopy (CLSM) image of FITC labeled insulin dissolved in sunflower oil (SO);

FIG. 4c is a confocal-laser-scanning-microscopy (CLSM) image of FITC labeled insulin dissolved in sunflower oil (SO) in the presence of CMC;

FIG. 4d is a confocal-laser-scanning-microscopy (CLSM) image of FITC labeled insulin dissolved in sunflower oil (SO) in the presence of CMC-6;

FIG. 4e is a confocal-laser-scanning-microscopy (CLSM) image of FITC labeled insulin dissolved in sunflower oil (SO) in the presence of CMC-12;

FIG. 5a is a photograph of a Franz cell apparatus illustrating skin processing (top), efficacy testing (lower left) and safety assay (lower right);

FIG. 5b is a histogram of insulin absorption [mU/L] as a function of time in minutes for as a function of wavelength [nm] for sunflower oil (SO), CMC, CMC-6 and CMC-12;

FIG. 5c is a bar graph of epidermal viability [MTT; percent of control] for control [100%], SDS, sunflower oil (SO), CMC, CMC-6 and CMC-12;

FIG. 5d is a bar graph IL-1a secreted in culture [ELISA pg/ml] for control, SDS, sunflower oil (SO), CMC, CMC-6 and CMC-12;

FIG. 5e is a series of photomicrographs depicting histology of the morphology of the untreated human skin (control) relative to skin treated with SDS, SO, CMC, CMC-6 and CMC 12;

FIG. 6a is a histogram of cumulative insulation permeation (%) as a function of time (Hrs.) for human skin treated with insulin in SO, CMC, CMC-6 and CMC-12;

FIG. 6b is a bar graph of residual insulin [% in donor chamber] for insulin in SO, CMC, CMC-6 and CMC-12; and

FIG. 6C is a bar graph of mass balance (insulin percentage) for insulin in SO, and CMC-12 showing distribution in unaccounted, transdermal permeation, viable skin, stratum corneum and donor.

### DETAILED DESCRIPTION OF EMBODIMENTS

Embodiments of the invention relate to transdermal delivery systems, methods of treatment, compositions for use in treatment of diabetes, and methods of manufacture.

Specifically, some embodiments of the invention can be used for transdermal delivery of hydrophilic active agents with a molecular weight greater than 1000 Da, 2000 Da, 3000 Da, 4000 Da or intermediate or greater molecular weights, such as insulin.

The principles and operation of transdermal delivery systems, methods of treatment, compositions for use in treatment of diabetes, and methods of manufacture according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Exemplary Transdermal Delivery System

In some exemplary embodiments of the invention there is provided a transdermal delivery system including one or more N-alkylamidated CMCs (carboxymethylcellulose) a hydrophilic active agent and a lipophilic carrier. CMC-6 and CMC-12 are examples of N-alkylamidated CMCs. In some embodiments the hydrophilic active agent includes insulin. According to various exemplary embodiments of the invention the transdermal delivery system is provided in a form selected from the group consisting of a cream, a lotion, an ointment and a patch. In some exemplary embodiments of the invention, the one or more N-alkylamidated CMCs includes two or more N-alkylamidated CMCs. In some embodiments the two or more N-alkylamidated CMCs each have a different absorption profile relative to skin and/or release profile relative to the hydrophilic active agent. In some embodiments use of several different N-alkylamidated CMCs, each with different absorption profiles relative to skin and/or different release profiles relative to the active agent contributes to an ability of the system to achieve sustained release/delivery of the hydrophilic active agent over a period of time. In some embodiments one of the one or more N-alkylamidated CMCs includes CMC-12.

According to various exemplary embodiments of the invention the transdermal delivery system is provided in a sustained release configuration or an immediate release configuration. In some exemplary embodiments of the invention, a ratio between the active agent (e.g. insulin) and the N-alkylamidated CMCs delivery compounds contributes to a release profile of the system.

### Exemplary Treatment Method

In some exemplary embodiments of the invention there is provided a method of treatment including applying a hydrophilic active agent to a skin surface in a carrier including one or more N-alkylamidated CMCs and a lipophilic diluent. In some embodiments the hydrophilic active agent includes insulin. Alternatively or additionally, in some embodiments the composition is formulated as an item selected from the group consisting of a cream, a lotion, an ointment and a patch to be contacted with the skin. Alternatively or additionally, in some embodiments the one or more N-alkylamidated CMCs includes two or more N-alkylamidated CMCs. Exemplary reasons for using two or more N-alkylamidated CMCs are set forth hereinabove. Alternatively or additionally, in some embodiments the one or more N-alkylamidated CMCs includes CMC-12.

### Exemplary Composition for Use

In some exemplary embodiments of the invention there is provided a composition comprising insulin in a carrier including one or more N-alkylamidated CMCs and a lipophilic diluent for use in treatment of diabetes. In some exemplary embodiments of the invention, the applying includes contacting an item selected from the group consisting of a cream, a lotion, an ointment and a patch to the skin. Alternatively or additionally, in some embodiments the one or more N-alkylamidated CMCs includes two or more N-alkylamidated CMCs. Exemplary reasons for use of two or more N-alkylamidated CMCs are set forth hereinabove. Alternatively or additionally, in some embodiments the one or more N-alkylamidated CMCs includes CMC-12.

### Exemplary Method of Manufacture

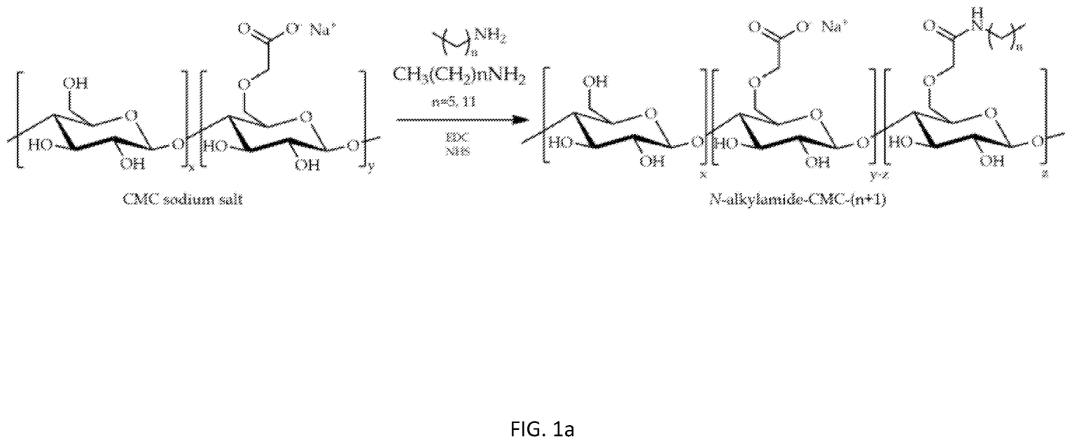

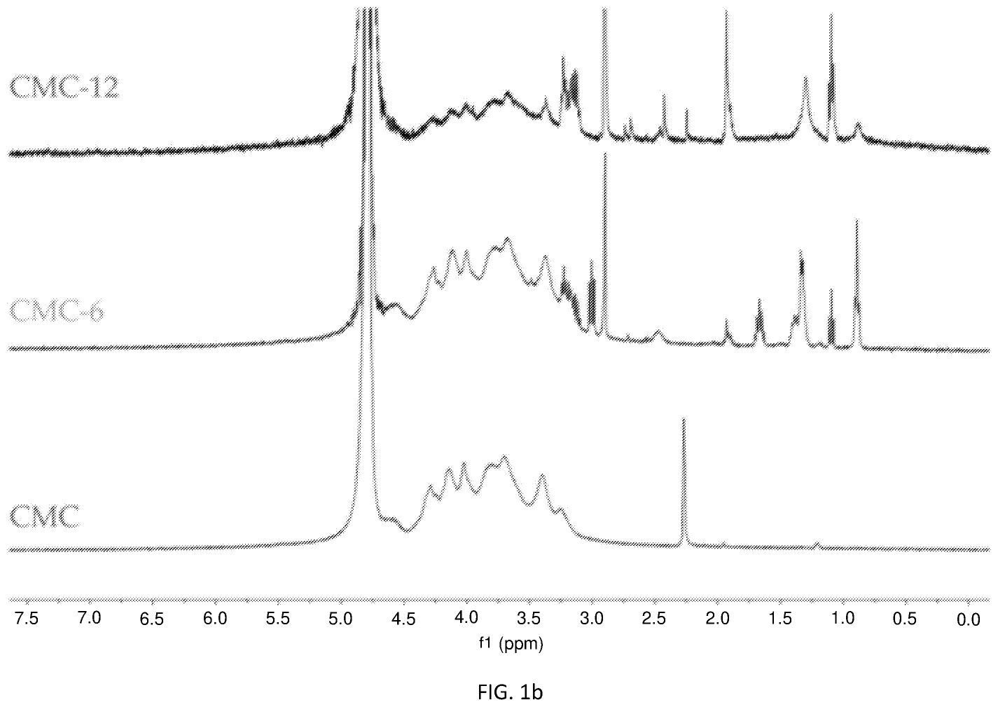

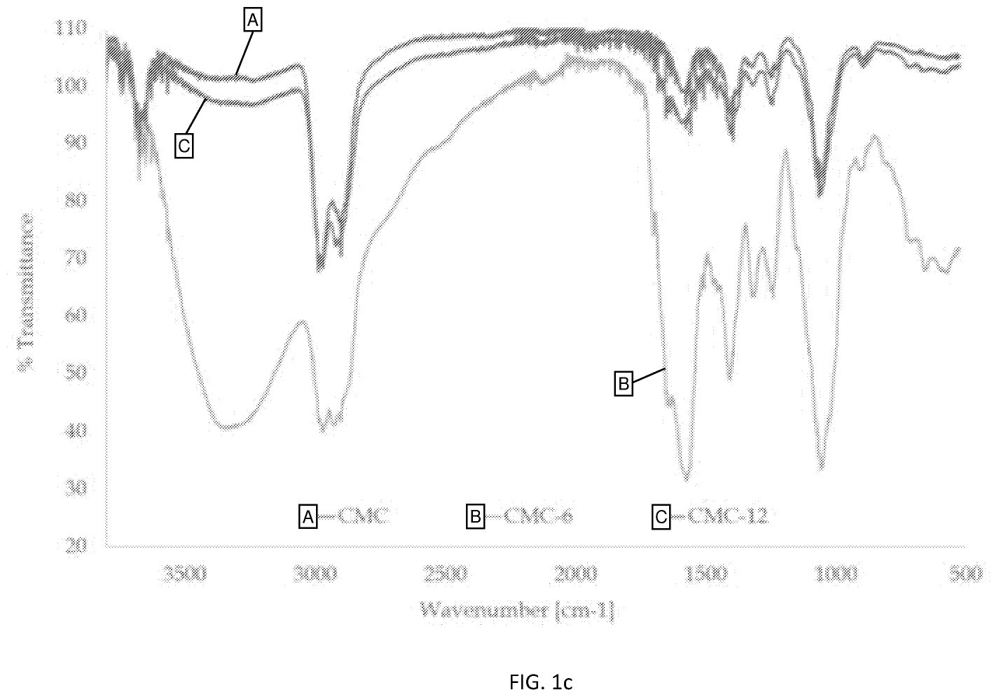

In some exemplary embodiments of the invention there is provided method of manufacture (summarized graphically in FIG. 1a) including dissolving carboxymethylcellulose (CMC) in water to form a CMC solution; dissolving 1-(3-Dimethylaminopropyl)-3-ethylcarbodiimide hydrochloride (EDC) and N-hydroxysuccinimide (NHS) in the CMC solution; and adding an amine to initiate N-alkylamidation of said CMC and produce a modified CMC solution. In some embodiments the amine includes hexyl amine and the N-alkylamidation produces CMC-6. Alternatively or additionally, in some embodiments the amine includes dodecyl amine and the N-alkylamidation produces CMC-12. Alternatively or additionally, in some embodiments the EDC and the NHS are provided in equimolar amounts. Alternatively or additionally, in some embodiments the EDC and the NHS and the amine are provided in equimolar amounts. Alternatively or additionally, in some embodiments the method includes dissolving a hydrophilic active agent in the modified CMC solution. In some embodiments the hydrophilic active agent comprises insulin. Alternatively or additionally, in some embodiments the method includes mixing a lipophilic diluent with the modified CMC solution.

It is expected that during the life of this patent many hydrophilic active agents will be developed and the scope of the invention is intended to include all such new technologies a priori.

As used herein the term “about” refers to +10%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and are not intended to limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

Alternatively or additionally, various exemplary embodiments of the invention exclude any specific feature, part, component, module, process or element which is not specifically disclosed herein.

Specifically, the invention has been described in the context of insulin but might also be used with other peptides or proteins.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

Additional objects, advantages, and novel features of various embodiments of the invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

### Materials and Methods

The following materials and methods are used in performance of experiments described in examples hereinbelow: Sodium carboxymethyl cellulose (MW=250 kDa; DS=0.9), n-dodecylamine and N-hydroxysuccinimide (NHS) were purchased from Acros Organics (Geel, Belgium).

n-Hexylamine pyrene, insulin (human recombinant) and insulin labeled with FITC (fluorescein isothiocyanate; human recombinant) were purchased from Sigma Aldrich (Steinheim, Germany).

1-(3-Dimethylaminopropyl)-3-ethylcarbodiimide hydrochloride (EDC) was purchased from Alfa Aesar (Lancashire, UK).

Dextran standards were purchased from PSS Polymer (Mainz, Germany).

Ethanol and ethanol absolute were purchased from Gadot Group (Netanya, Israel).

Water (HPLC grade) was purchased from Bio Lab (Jerusalem, Israel).

Organic cold-pressed sunflower oil (SO) was purchased from Joe & Co. (Vicenza, Italy).

Deionized water (DW) was obtained by mechanically filtering water through a Treion TS1173 column.

Deuterated solvent for the NMR analysis (D₂O) was purchased from Armar Chemicals (Döttingen, Switzerland).

All reagents and solvents were used without any further purification.

### Synthesis of CMC-6 and CMC-12

N-alkylamidated modifications were prepared by dissolving 0.58% (w/w) carboxymethyl cellulose (CMC) in 100 mL DW at 60° C. Once the solution achieved homogeneity, it was cooled down to room temperature and 1.3 mmol of EDC and 1.3 mmol of NHS were added. After 2.5 h of stirring, 1.3 mmol of a selected amine (hexyl amine or dodecyl amine) were added and the solution was stirred overnight (dodecylamine was first dissolved in 15 ml of absolute ethanol and then added to the reaction solution at 40° C.). According to the manufacturer, the commercial CMC's DS is 0.9, so amount of carboxymethyl groups [mole] available for N-alkylamidation was calculated as [(0.58 g/270 g/mol)*0.9], where 0.58 g is the mass of the CMC polymer, 270 is the MW of the CMC monomer and 0.9 is the monomer molar fraction. Amines, EDC and NHS were added at 0.7 eq in respect to CMC's carboxymethyl groups. The N-alkylamidated derivatives CMC-6 and CMC-12 were precipitated by adding six times the volume of ethanol (vs. the reaction solution volume), isolating the material via centrifugation, washing it with ethanol three times, and then drying it in a vacuum desiccator overnight. This synthesis protocol is summarized in FIG. 1a.

Attenuated total reflection-Fourier transform infrared (ATR-FTIR) spectroscopy:

ATR-FTIR spectroscopy was performed using a Thermo Scientific Nicolet iS5 FTIR spectrometer (USA). Modified CMC powders were subjected to 32 scans at a 0.5 cm⁻¹ resolution between 500 and 4000 cm⁻¹.

### Nuclear Magnetic Resonance (NMR)

¹H NMR spectra were recorded using Bruker Avance I and Avance III NMR 400 MHZ spectrometers (USA). Chemical shifts are reported in parts per million (ppm). 1H-NMR spectra were calibrated to the solvent residual peak (H₂O at 4.79 ppm). All of the NMR samples were prepared using D₂O as a solvent at 298K. The original CMC peaks were found to be in accordance with those reported in previous publications (Kono et al. (2016) Carbohydr. Polym. 146:1-9).

### Degree of Substitution:

The total organic carbon (TOC) and the total nitrogen (TN) contents of CMC, CMC-6 and CMC-12 were determined in water by high-temperature catalytic combustion and chemiluminescence detection using a Shimadzu (USA) ASTM D 8083 analyzer elemental analyzer (USA, TOC-L with TNM). CMC, CMC-6 and CMC-12 powders were dissolved in distilled water to a final concentration of 0.2 mg/mL. The percentage of CMC substitution by hexyl/dodecyl amide was calculated using the following equation:

$$\% Subsitution = \frac{100 * \frac{TOC [ CMC ]}{Mw [ C ]}}{\frac{TN [ CMC 6 ∨ CMC 12 ]}{Mw [ N ]}}$$

### Gel-Permeation Chromatography (GPC):

The molecular weights and polydispersity indices of CMC, CMC-6 and CMC-12 were determined using gel-permeation chromatography (GPC). Waters' Alliance system e2695 separations module was used (Waters, USA), equipped with a refractive index detector, model Blue 2414. The mobile phase was HPLC-grade water under isocratic elution for 30 min at a flow rate of 0.7 mL/min. The injection volume was 20 μl and the temperature of both the detector and the columns was 30° C. Analyses were carried out using an ultra-hydrogel column: 1000 Å, 12 μm, 7.8 mm×300 mm, 2-4,000 kDa (Waters, USA). The molecular weights were determined according to a dextran standards kit with a number average molecular weight [Mn] range of 3,300-333,000 Da (PSS Polymer Standards Service GmbH, Germany). All data provided by the GPC system were collected and analyzed with the Empower 3 personal dissolution software. CMC, CMC-6 and CMC-12 powders were dissolved in the mobile phase to a final concentration of 1 mg/mL. The solutions were filtered through a 0.22-μm nylon syringe filter.

### Thermogravimetric Analysis (TGA):

Thermogravimetric analysis (TGA) was performed using a Perkin-Elmer TGA 8000 device (TA Instruments, USA). Ceramic crucibles were loaded with 1-3 mg of each sample and heated from 50° C. to 800° C. at a rate of 10° C./min under the flow of a N₂ atmosphere (20 mL/min).

### Differential Scanning Calorimetry (DSC):

Differential-scanning calorimetry (DSC) measurements were conducted with a Perkin-Elmer DSC 6000 instrument (USA) calibrated using indium and zinc standards. Thermograms of each sample were obtained from the second heating run up to 440° C., after the first run of heating up to 160° C. and cooling to 50° C. at a constant rate of 20° C./min, under a N₂ purge of 20 mL/min. Aluminum crucibles with pierced lids were loaded with 5-15 mg of each sample.

### Critical-Aggregation-Concentration Measurements:

The critical aggregation concentrations of CMC-6 and CMC-12 were studied using the pyrene-fluorescent-probe method [39]. The ratio between two specific peaks (i.e., I₃˜383 nm and I₁˜373 nm) in pyrene's spectrum was used as a quantitative measurement for the aggregation point. The diluent was prepared by adding 25 μl of the pyrene stock solution (0.49 mg/mL) to 50 ml of distilled water to give a final concentration of 1.2 μM. A 15 mg/mL CMC-6 solution or 1.5 mg/mL CMC-12 solution was dissolved in the diluent and the mixture was stirred overnight. Then, the prepared solutions were repeatedly diluted by a factor of 2 with the abovementioned diluent. 150 μL of each solution were loaded onto a 96-well plate. The fluorescence emission intensity was scanned for each well using a SynergyHTX multi-mode reader device (BioTek Instruments, USA). The excitation wavelength for pyrene is 340 nm and the emission band was recorded at 360-400 nm, with a resolution of 1 nm. All samples measured were kept at 26±1° C. All measurements were done in triplicate. Critical-aggregation-concentration values were calculated as the intersection between two linear lines depicting aggregate formation dependent on concentration in solution.

### Transmission Electron Microscopy (TEM):

Samples (3 μl drop) of aqueous solutions of CMC-6 and CMC-12 (CMC-6:20 mg/ml; CMC-12:1.5 mg/mL) were placed on glow-discharged TEM grids (ultrathin carbon on carbon lacey support film, 400-mesh copper grids, Ted Pella, Inc.). The excess liquid was blotted with filter paper and the grids were allowed to dry in the air. The samples were examined by a FEI Tecnai 12 G2 TWIN TEM operated at 120 kV. Images were recorded using 4 k×4 k FEI Eagle CCD camera (ThermoFisher Scientific, USA).

### Solubilization of CMCs in Sunflower Oil (a Lipophilic Medium):

Aqueous CMC, CMC-6 and CMC-12 solutions (0.5 mL each) at a concentration of 25 mg/ml were added to 10 ml of sunflower oil. The resulting solutions were then heated to 100° C. with stirring, to evaporate any water and to yield a final polymer concentration of 1.25 mg/ml of the oil.

In vitro evaluation of CMC-6 and CMC-12 as vehicles to introduce insulin into a lipid environment:

In order to evaluate the capability of CMC-6 and CMC-12 to insert insulin into a lipid environment an in vitro fluorimetry study was performed. FITC labeled insulin (was added to sunflower-oil solutions of the CMC derivatives prepared as described above to reach an insulin-FITC concentration of 1 mg/ml and stirred for 48 h, while the solutions were kept covered with aluminum foil. The prepared solutions were kept for seven days at ambient temperature covered by aluminum foil for gravitational filtration. After the seven days, 100 μL of each solution were loaded onto a 96-well plate. The fluorescence emission intensity was scanned for each well using a Synergy-neo2 multi-mode reader device (BioTek Instruments, USA). The excitation wavelength for FITC was 490 nm and the emission band recorded was 510-700 nm, at increments of 5 nm. The florescent emission of insulin-FITC added to sunflower oil that did not contain any CMC was used as a control.

### Encapsulation Studies—Confocal Laser Scanning Microscopy (CLSM):

FITC labeled insulin was added to the sunflower-oil solutions of the CMC derivatives prepared as described above to reach an insulin-FITC concentration of 0.5 mg/ml. The mixtures were stirred for 48 h, while they were kept covered by aluminum foil. The prepared solutions were kept for a week at ambient temperature, covered by aluminum foil, for gravitational filtration. About 20 μL of each solution were loaded onto microscope slide. Images were acquired using a Leica SP8 laser scanning microscope (Leica, Wetzlar, Germany), equipped with an OPSL 488-nm laser, HC PL APO CS2 63×/1.20 water objective (Leica, Wetzlar, Germany) and Leica Application Suite X software (LASX, Leica, Wetzlar, Germany). The FITC emission signal was detected with a HyD (hybrid) detector in the range of 500-550 nm.

### Ex Vivo Transdermal Delivery Studies:

Transdermal insulin delivery was assessed by the Franz cell apparatus (PermeGear, PA, USA). Human skin (full thickness) was obtained from Zen-bio (NC, US). A mechanical skin press apparatus was used to section the skin and it was kept at −20° C. until it was used. On the day of experiment, the skin samples were pre-equilibrated with the PBS for 1 h prior to use, with the dermal side submerged in the buffer. Then, the skin samples were clamped between the donor and receptor chambers, with the epidermal side facing the donor compartment. The donor chamber was loaded with 200 μL of the sunflower-oil solutions of the biopolymers (CMC-6 and CMC-12), all containing insulin (0.2 mg/ml). The sunflower oil containing insulin without biopolymer was used as a control treatment. The receptor chamber was filled with 5 ml of PBS containing sodium azide (to prevent microbial growth) and kept at 32±0.5° C. under constant stirring (500 rpm). Samples (200 μL each) were taken at the indicated time points and an equal volume of the buffer was added immediately. The amount of insulin permeation was quantified by ELISA, using a designated standard curve [Mercodia]. All of the samples were tested in three repetitions.

### Skin Viability and Irritation:

All cell-culture media and reagents were purchased from Biological Industries (Beit-HaEmek, Israel). The skin samples were obtained from healthy women (between 30 and 65 years old) who were undergoing aesthetic abdominal surgery and had signed an informed-consent form. The experiments were conducted with the approval of the IRB (Helsinki Committee) of Soroka Medical Center, Beer Sheva, Israel. Human skin culture preparation and treatments were performed under aseptic conditions. A mechanical skin-press apparatus was used to section the skin into 0.64-cm² pieces, as previously described (Ogen-Shtern et al. (2020) J. Cosmet. Dermatol. 19:1522-1527). The skin explants were maintained in an air-liquid interface, with the dermal side submerged in the liquid. The biopolymers were applied topically (3 μl). After 48 h, the spent media was discarded and IL-1α was evaluated by ELISA (Biolegend, CA, US). In addition, epidermis was separated and viability was determined by MTT, as previously reported after 48 h (Kahremany, et al. (2010) Skin Pharmacol. Physiol. 32:173-181). For morphological evaluation, the samples were fixed with 4% formaldehyde for 1 h at room temperature. Then, the tissues were washed with PBS and kept in 70% ethanol at 2-8° C. until use. Following dehydration, paraffin sections (10 μm) were prepared and slides were stained with hematoxylin-eosin solution.

### Example 1

### Synthesis of N-Alkylamidated Carboxymethyl Cellulose (CMC)

In order to provide a supply of modified polysaccharide for testing as a potential insulin delivery vehicle, N-alkylamidated polysaccharides were synthesized in a one-step reaction of carboxymethyl cellulose (CMC) with hexylamine or dodecylamine to yield CMC-6 or CMC-12, respectively (FIG. 1). The isolated products were characterized by ATR-FTIR and 1H-NMR spectroscopy, which confirmed the successful coupling with no traces of reactants. The ATR-FTIR scans included new peaks for alkyl C—H bending at 680-720 cm⁻¹, amide frequencies at 1560-1580 cm⁻¹ and an N—H stretch at ˜3300 cm⁻¹. The 1H-NMR scans showed the aliphatic protons of the coupled alkyl chains at 0.8-2.1 ppm and the α-to-amide-bond protons at 2.8-3.2 ppm.

FIG. 1a is a diagrammatic representation of the Synthesis of N-alkylamidated carboxymethyl cellulose derivatives, referred to as CMC-6 and CMC-12.

FIG. 1b is histogram of 1H-NMR 400 MHz in arbitrary units as a function of f1(ppm) for CMC; CMC-6 and CMC-12 as indicated

FIG. 1c is histogram of ATR-FTIR in % Transmittance as a function of Wavenumber [cm⁻¹] for CMC; CMC-6 and CMC-12 as indicated.

### Example 2

### Thermal Gravimetric Analysis (TGA) and Differential Scanning Calorimetry of N-Alkylamidated CMC

CMC-6 and CMC-12 as prepared in Example 1 were subjected to Thermal gravimetric analysis and differential scanning calorimetry as described above.

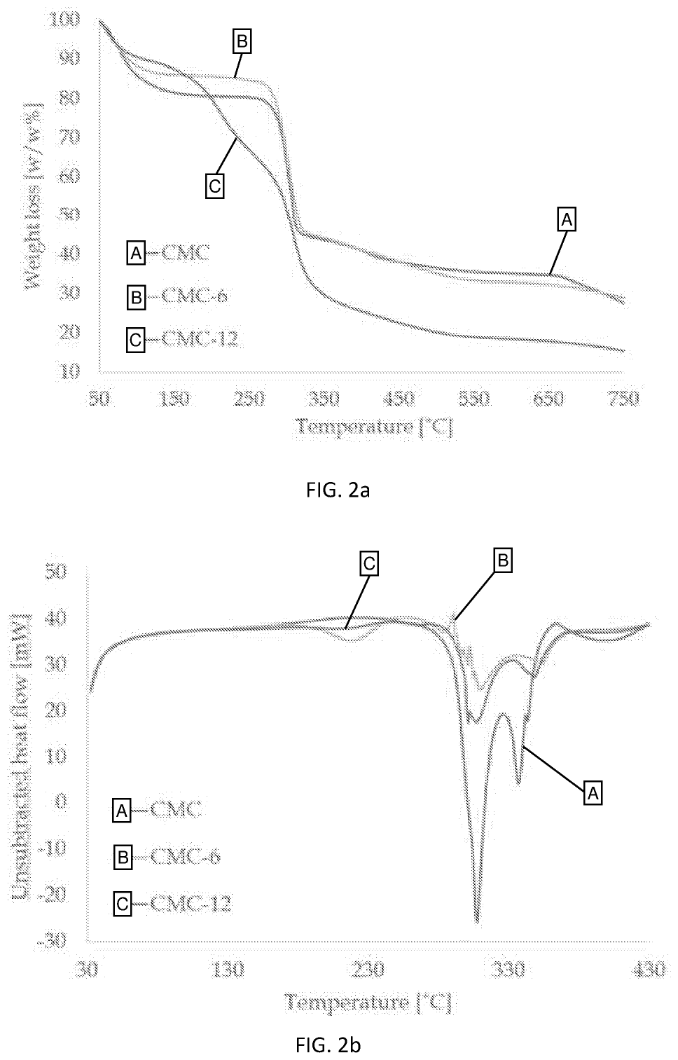

Thermal gravimetric analysis (TGA) (FIG. 2a) and differential scanning calorimetry (DSC) (FIG. 2b) studies of the modified CMC-6 and CMC-12 were performed and compared to unmodified CMC.

CMC-6 displayed a thermal decomposition pattern similar to that of the original CMC with a water-weight-loss event at ˜ 57° C. and main pyrolytic decomposition events at 286.0 and 283.5° C. for CMC and CMC-6, respectively.

The dodecyl-substituted CMC-12 spectra was different, showing two defined pyrolytic-decomposition events, one at 187.3° C. (with 20.3% weight loss) and the other at 290.0 (with 38.5% weight loss).

The total weight-loss for CMC, CMC-6, and CMC-12 were 77.6%, 71.9% and 87.2%, respectively. DSC studies of the polysaccharides revealed thermal-decomposition temperatures that were higher than 300° C., without any prior glass transition, due to the polysaccharides' semi-rigid backbones.

### Example 3: Degree of Substitution of CMC-6 and CMC-12

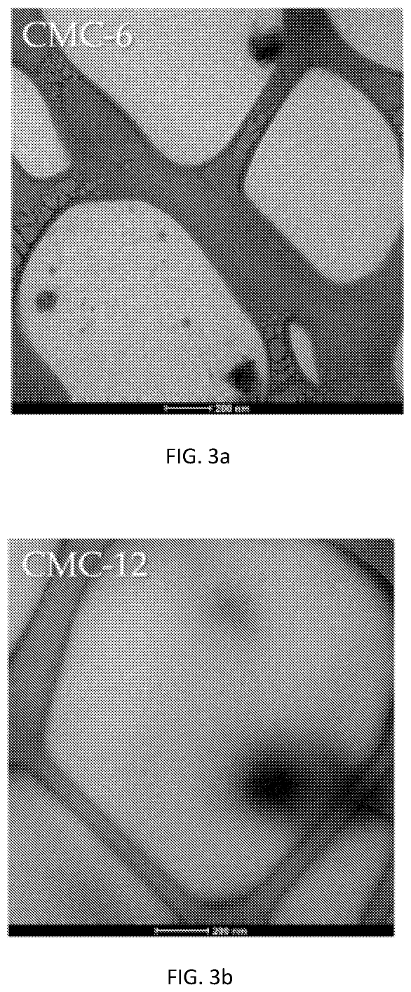

The degree of substitution was determined using the total-organic-carbon (TOC) method and was found to be 24% and 18% for CMC-6 and CMC-12, respectively. Conjugation with aliphatic amines provides the modified polysaccharides with amphiphilic properties, allowing spontaneous self-assemble that takes place above a specific concentration, termed the critical aggregation concentration (Table 1). Whereas the original CMC does not undergo self-assembly, CMC-6 and CMC-12 derivatives spontaneously assemble at concentrations above 1.66 and 0.05 mg/mL, respectively. Transmission electron microscopy (TEM) studies confirmed the formation of stable nanometric aggregates, 50-150 nm in size for CMC-6 (FIG. 3a) and 150-230 nm in size for CMC-12 (FIG. 3b).

Gel-permeation chromatography (GPC) revealed that the coupled aliphatic chains caused a decrease in the retention time; the longer the chain, the shorter the retention time. The number average molecular weight (Mn) of the prepared polymers was calculated using dextran as a calibrating standard. Mn increases with the length of the coupled chain. It should, however, be noted that that Mn is better to be viewed as a comparing parameter and not as an absolute value. The spontaneous assembling ability changes the spatial structure of the prepared CMC-6 and CMC-8, which affects their gel-permeation properties and Mn values (Table 1).

TABLE 1
Degree of substitution (DS), Critical aggregation concentration
(CAC), retention time (RT), number average molecular weight
(Mn) and polydispersity (PDI) for CMC, CMC-6and CMC-12.

| | DS | CAC | RT | Mn | |
|---|---|---|---|---|---|
| | [%] | [mg/mL] | [min] | [kDa] | PDI |
| CMC | — | — | 10.397 | 154 | 1.97 |
| CMC-6 | 24 | 1.66 ± 0.58 | 9.486 | 584 | 2.95 |
| CMC-12 | 18 | 0.05 ± 0.004 | 8.653 | 5423 | 2.85 |

### Example 4

### In Vitro Assays of Insulin Solubility in a Lipid Environment

The ability of CMC-6 and CMC-12 biopolymers to increase solubility of insulin in a lipid environment was studied in order to evaluate the possibility of using these polymers as a transdermal delivery system for insulin.

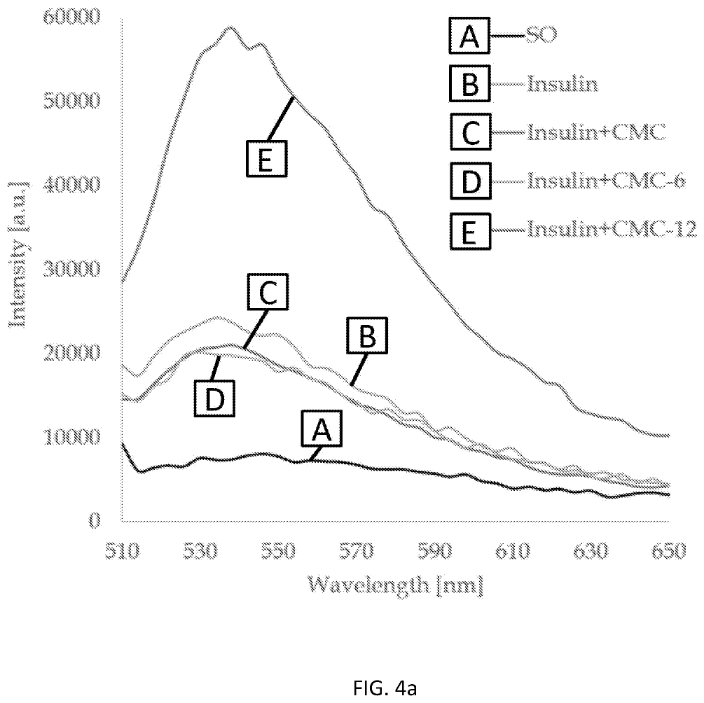

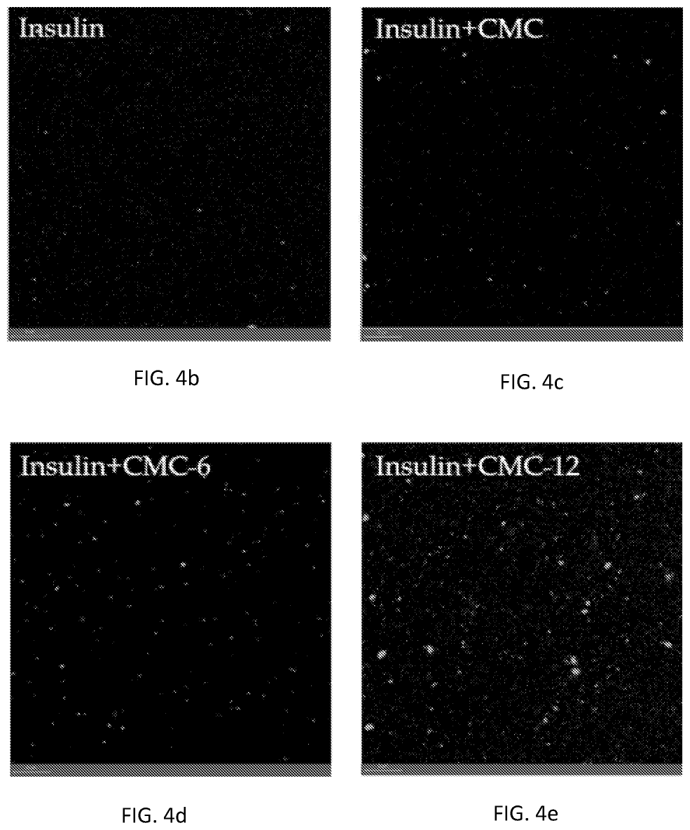

First, in vitro studies of the abilities of CMC-6 and CMC-12 to introduce insulin into a lipid environment were performed. For this purpose, FITC labeled insulin was used and the fluorescence emission of the labeled insulin in sunflower oil with and without carriers was measured using a fluorimeter. Results summarized in FIG. 4a indicate that FITC labeled insulin produced the most fluorescence in vitro in the presence of CMC-12.

FIG. 4b is a confocal-laser-scanning-microscopy (CLSM) image of FITC labeled insulin dissolved in sunflower oil (SO).

FIG. 4c is a confocal-laser-scanning-microscopy (CLSM) image of FITC labeled insulin dissolved in sunflower oil (SO) in the presence of CMC.

FIG. 4d is a confocal-laser-scanning-microscopy (CLSM) image of FITC labeled insulin dissolved in sunflower oil (SO) in the presence of CMC-6.

FIG. 4e is a confocal-laser-scanning-microscopy (CLSM) image of FITC labeled insulin dissolved in sunflower oil (SO) in the presence of CMC-12.

These CLSM image of FIG. 4e confirms visually the results of the fluorimetry assay summarized in FIG. 4a.

In summary, CMC-12 carrier substantially enhanced the solubility of insulin in sunflower oil. In addition, the ability of the prepared nanoaggregates to carry insulin in a lipid environment was confirmed CLSM (FIG. 4e)

### Example 5

### Ex Vivo Assays of Insulin Penetration of Skin

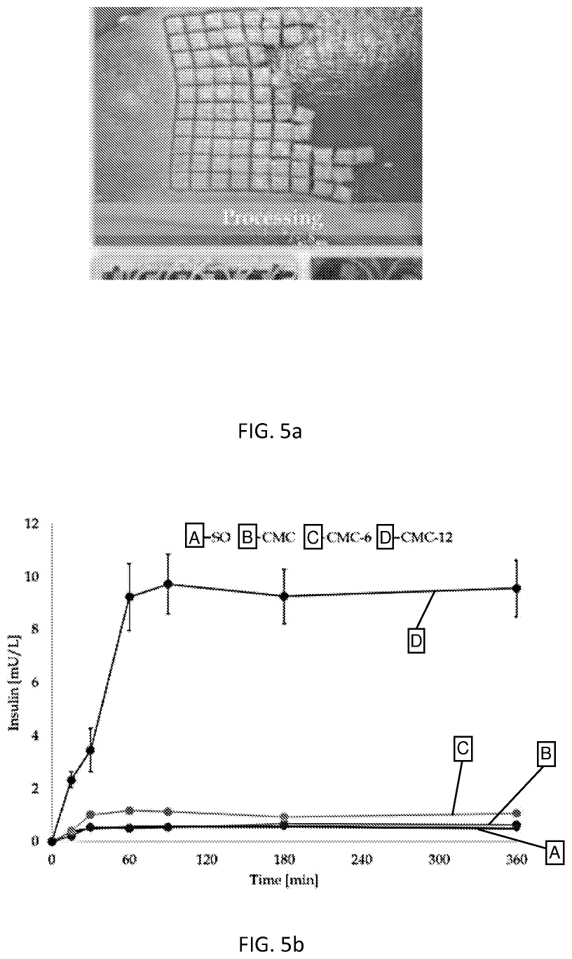

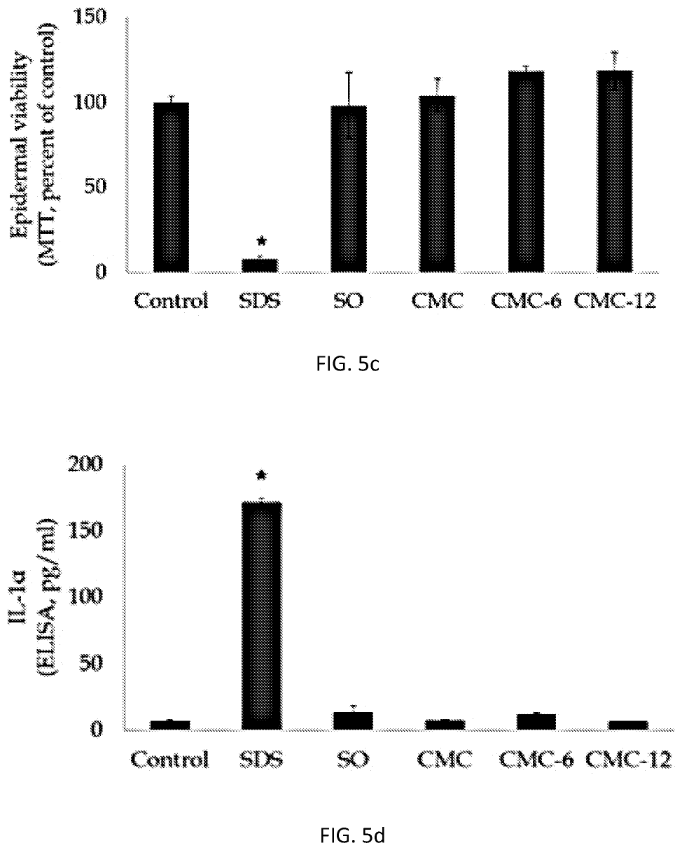

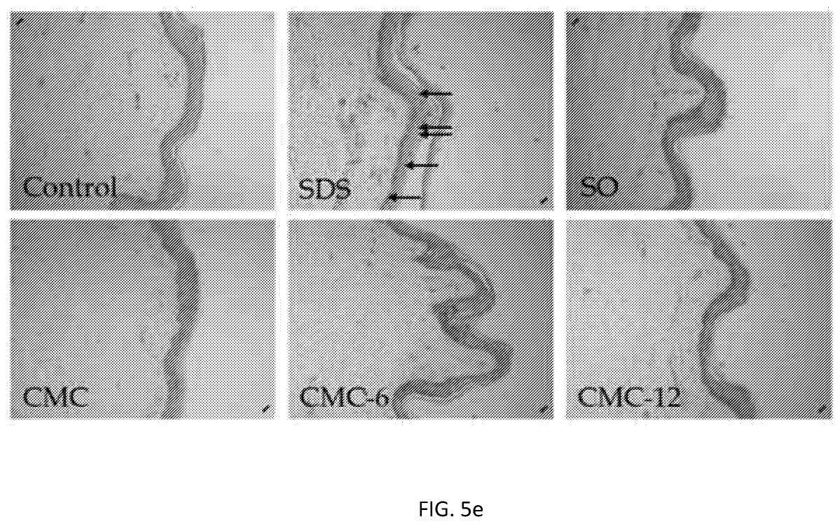

In order to ascertain whether the ability of CMC-6 and CMC-12 to increases solubility of insulin in lipids translates to an ability to use these polymers for transdermal delivery, the insulin-carrying biopolymers were mounted on a Franz cell apparatus (FIG. 5a). The kinetics of their permeation across human skin ex vivo are summarized in FIG. 5b.

As shown in FIG. 5b, the unmodified CMC had no noticeable impact and did not enhance insulin permeation. CMC-6 slightly enhanced insulin permeation. CMC-12 as an insulin carrier produced a massive increase in insulin permeation, correlating with the results observed in the in vitro studies of Example 4. It is possible that the superior performance of CMC-12 can be attributed to a long dodecyl substituent that increases the membrane-penetration ability of that carrier. In addition, it can be speculated that dodecyl-substituted CMC-12 biopolymer facilitates the formation of less tightly self-assembled structures, making it a better choice for the encapsulation of large macromolecules such as insulin.

Next, an ex vivo human skin organ culture (FIG. 5a; lower right panel) was used to exclude the possibility of skin damage during the transdermal delivery process. The insulin-containing biopolymers were topically applied to the epidermal side of the skin. As expected, sodium dodecyl sulfate (SDS) markedly compromised skin viability, as seen in the MTT results of FIG. 5c. However, none of the biopolymers had any negative effects on the skin, in comparison to the naïve untreated control (FIG. 5c). Similar results were obtained from the histological evaluations: SDS markedly disrupted the epidermal layer; whereas no morphological alterations by the vehicle (SO) or biopolymers were recorded. Lastly, the secreted level of the irritation cytokine IL-1α was used as an independent marker to assess the impact of the biopolymers on the skin. Results summarized in FIG. 5d clearly showed no increase in secretion of IL-1a from CMC, CMC-6 or CMC-12. Thus, these biopolymers are verified as both effective for transdermal insulin delivery and safe to use in a transdermal system.

FIG. 5e is a series of photomicrographs depicting histology of the morphology of the untreated human skin (control) relative to skin treated with SDS, SO, CMC, CMC-6 and CMC 12. Arrows in the SDS panel indicate tissue damage, confirming the MTT results of FIG. 5c and the IL-1a result of FIG. 5d.

In summary this example shows that CMC-12 is a safe and effective carrier for transdermal delivery of insulin or other peptides/proteins.

### Example 6

### Trans-Epidermal Water Loss (TEWL) and Insulin Delivery

Skin integrity was evaluated by trans-epidermal water loss (TEWL) before and after application of SO, CMC, CMC-6 and CMC-12. The baseline readings were in the expected range and no significant increase was seen after 5 hrs. or 24 hrs. of exposure. A solution of 10% SDS (used as positive control) increased water loss by almost 3-fold in the same experimental settings. Results are summarized in Table 2.

TABLE 2
trans-epidermal water loss (TEWL) in g/m²/hr

| | SO | CMC | CMC6 | CMC12 | 10% SDS |
|---|---|---|---|---|---|
| Baseline | 9.6 ± 0.79 | 9.3 ± 0.63 | 8.1 ± 0.38 | 9.7 ± 0.19 | 10.0 ± 2.4 |
| Post exposure | 10.1 ± 2.7 | 9.2 ± 0.67 | 7.0 ± 1.76 | 9.1 ± 1.7 | |
| (5 hr) | | | | | |
| Baseline | 9.8 ± 0.68 | 9.7 ± 1.37 | 9.7 ± 0.28 | 10.6 ± 0.51 | |
| Post exposure | 10.0 ± 0.45 | 10.1 ± 0.33 | 10.0 ± 0.22 | 9.7 ± 0.76 | |
| (24 hr) | | | | | |

Methylene blue dye was also used to monitor skin integrity following application of SO, CMC, CMC-6 and CMC-12. Results summarized in Table 3 confirm the TEWL results presented in Table 2.

TABLE 3
Methylene blue μg/ml

| | 5 hr | 24 hr |
|---|---|---|
| SO | <0.01 | <0.01 |
| CMC | <0.01 | <0.01 |
| CMC6 | <0.01 | <0.01 |
| CMC12 | <0.01 | <0.01 |
| 10% SDS | 0.13 ± 0.06 | |

Taken together with the evaluation of skin morphology by H&E staining (FIG. 5e) and viability results (FIG. 5C), the TEWL and Methylene blue results confirm that the insulin permeation is not because of damage to the skin.

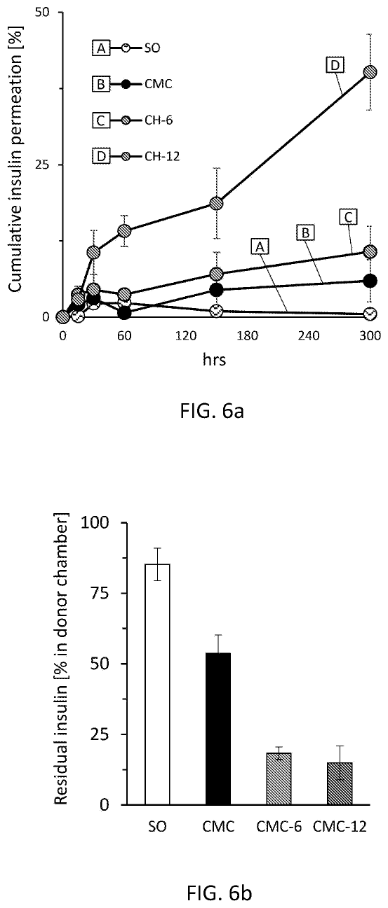

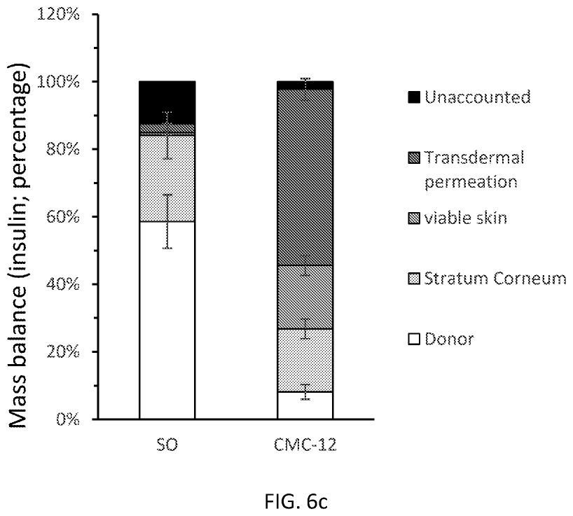

Increasing the concentration of insulin in the donor compartment in the Franz cell apparatus prolonged insulin permeation, as seen FIG. 6a. This was accompanied by a corresponding decrease in the residual insulin levels in the donor compartment as seen in FIG. 6b which shows a reduction in insulin of over 75% in the CMC-6 and CMC-12 treated groups. FIG. 6c is a bar graph of mass balance of CMC-12 relative to the SO carrier. Collectively, this experiment shows that when insulin levels are increased, CMC-6 and especially CMC-12 can enhance insulin transdermal permeation as well as insulin level throughout the stratum corneum. In addition, the high level of insulin used here can result in increased insulin permeation time, suggesting that CMC-6 and/or CMC-12 can be used for sustained release of insulin over time.

1. A transdermal delivery system comprising:
a) one or more N-alkylamidated CMCs (carboxymethylcellulose);
b) a hydrophilic active agent; and
c) a lipophilic carrier.

2. A transdermal delivery system according to claim 1, wherein said hydrophilic active agent comprises insulin.

3. A transdermal delivery system according to claim 1, provided in a form selected from the group consisting of a cream, a lotion, an ointment and a patch.

4. A transdermal delivery system according to claim 1, provided in a sustained release configuration.

5. A transdermal delivery system according to claim 1, provided in an immediate release configuration.

6. A transdermal delivery system according to claim 1, wherein said one or more N-alkylamidated CMCs includes two or more N-alkylamidated CMCs.

7. A transdermal delivery system according to claim 1, wherein said one or more N-alkylamidated CMCs includes CMC-12.

8. A method of treatment comprising:
applying a hydrophilic active agent to a skin surface in a carrier including one or more N-alkylamidated CMCs and a lipophilic diluent.

9. A method of treatment according to claim 8, wherein said hydrophilic active agent comprises insulin.

10. A method of treatment according to claim 8, wherein said applying includes contacting an item selected from the group consisting of a cream, a lotion, an ointment and a patch to the skin.

11. A method of treatment according to claim 8, wherein said one or more N-alkylamidated CMCs includes two or more N-alkylamidated CMCs.

12. A method of treatment according to claim 8, wherein said one or more N-alkylamidated CMCs includes CMC-12.

13-16. (canceled)

17. A method of manufacture comprising:
(a) dissolving carboxymethylcellulose (CMC) in water to form a CMC solution;
(b) dissolving 1-(3-Dimethylaminopropyl)-3-ethylcarbodiimide hydrochloride (EDC) and N-hydroxysuccinimide (NHS) in said CMC solution; and
(c) adding an amine to initiate N-alkylamidation of said CMC and produce a modified CMC solution.

18. A method according to claim 17, wherein said amine includes hexyl amine and said N-alkylamidation produces CMC-6.

19. A method according to claim 17, wherein said amine includes dodecyl amine and said N-alkylamidation produces CMC-12.

20. A method according to claim 17, wherein said EDC and said NHS are provided in equimolar amounts.

21. A method according to claim 20, wherein said EDC and said NHS and said amine are provided in equimolar amounts.

22. A method according to claim 17, comprising dissolving a hydrophilic active agent in said modified CMC solution.

23. A method according to claim 22, wherein said hydrophilic active agent comprises insulin.

24. A method according to claim 17, comprising mixing a lipophilic diluent with said modified CMC solution.
